(19) United States
(12) Patent Application Publication
(10) Pub. No.: US 2026/0308644 A1
(43) Pub. Date: Oct. 8, 2026

(54) BIOETHANOL PRODUCTION FROM WASTE USING ENZYMES

(71) Applicant: Philip HALL, Sevenoaks (GB)

(72) Inventor: Philip HALL, Sevenoaks (GB)

(21) Appl. No.: 19/481,585

(22) PCT Filed: May 2, 2024
(86) PCT No.: PCT/GB2024/051157

§ 371 Date: Nov. 5, 2025

(30) Foreign Application Priority Data
May 5, 2023 (GB) 2306704.4

(51) Int. Cl. C12P 7/10

(52) U.S. Cl. CPC C12P 7/10

(57) ABSTRACT

The invention relates to a method for treating waste material. The method comprises the steps of: a. converting the waste material into a slurry, e.g., by adding water; b. adding enzymes to the slurry and allowing the enzymes to extract sugars from the slurry by enzyme saccharification; c. separating the liquid content of the slurry from the solid waste content to give a liquid; d. converting the liquid to an ethanol-water mixture; and e. concentrating or separating the ethanol-water mixture to give ethanol. An apparatus for treating waste material according to the method is also provided.

The present invention relates to the processing of waste material into bioethanol and more particularly to the processing of municipal domestic waste, also known as municipal solid waste (MSW) into bioethanol.

### BACKGROUND

Globally, thousands of tonnes of MSW are generated annually, about 10% of which from the UK and the EU. Landfilling, open dumping, recycling, composting and incinerating MSW has contributed significantly to greenhouse gas emissions. Indeed, incinerating waste usually creates sulphur emissions and requires high unsightly chimneys and incinerators are inefficient requiring high energy inputs. Authorities are becoming increasingly more reluctant to grant licenses for new incinerators and are closing landfill sites. International climate change targets have stated that greenhouse gas emissions must be cut by 60% by 2050.

Previous methods of converting biomass into ethanol have relied on acid hydrolysis, e.g., JP2006088136A, WO2010146243A1, EP2971028B1, JP2009112246A, WO2014041373A2, WO2009095693A2, and WO2008025522A1. Acid hydrolysis typically uses hydrochloric acid, sulfuric acid, trifluoroacetic acid, formic acid, or nitric acid to convert cellulose and hemicelluloses into sugar and lignin by breaking down the cellulose to produce hexose and pentose sugars leaving behind lignin. The acid-sugar solution is separated, typically by means of ion exchange technology, which separates the components without diluting the sugars. The acid may then be recycled and any acid in the sugar solution is neutralised. The sugar solution may then be fermented to give ethanol. The use of highly corrosive acids is necessary in acid hydrolysis but makes the process hazardous and environmentally unfavourable. Thus, there is a need for alternative methods of converting MSW into bioethanol.

In response to this need, the inventor has devised an innovative apparatus and method of achieving eco-friendly and financially viable bioethanol using a blend of enzymes.

### SUMMARY

The present invention provides an apparatus and method for processing municipal waste into bioethanol. The present invention is energy efficient and environmentally friendly.

### DESCRIPTION

The invention relates to a method for treating waste material comprising the steps of:
a. converting the waste material into a slurry, e.g., by adding water;
b. adding enzymes to the slurry and allowing the enzymes to extract sugars from the slurry by enzyme saccharification;
c. separating the liquid content of the slurry from the solid waste content to give a liquid;
d. converting the liquid to an ethanol-water mixture; and
e. concentrating or separating the ethanol-water mixture to give ethanol.

The waste material may be processed into a slurry in step a., wherein the particle size is reduced to less than about 10 mm, optionally less than about 5 mm, preferably about 1-3 mm.

Prior to step b. the slurry may be heated to above the boiling point of water; for instance, to 100 to 175° C., optionally 125 to 150° C., preferably about 138° C., to assist in breaking down the slurry.

Prior to step b. the slurry may be heated to 60 to 125° C., optionally 70 to 100° C., preferably about 85° C., to assist in breaking down the slurry.

The temperature of the slurry may be increased or reduced to about 40-42° C., optionally using heat exchangers, prior to adding the enzymes in step b.

The pH of the slurry may be adjusted to suit the enzymes, for instance to pH 5, before, during or after adding the enzymes.

In step c. the liquid content of the slurry may be separated from the solid waste content using a centrifuge.

In step d. the liquid may be converted into an ethanol-water mixture using yeast, e.g., using a turbo-yeast.

The CO₂ generated in step d. during the conversion of the liquid to an ethanol-water mixture may be captured. The captured CO₂ may be used during subsequent methods to prepare carbonic acid which may be used to lower the pH during subsequent methods and assist in breaking down the slurry following step a. Alternatively, the method may use CO₂ captured during an earlier method to prepare carbonic acid, which may be used to lower the pH and assist in breaking down the slurry following step a.

In step e. the ethanol-water mixture may be concentrated using distillation and/or a zeolite column.

The enzyme saccharification in step b. may be allowed to occur over 5-10 hours; and/or at 40-42° C., and/or at pH 5.

The solid waste content obtained in step c. may be used as fuel to heat the process during subsequent methods. Alternatively, the solid waste content generated during an earlier method may be used to heat the process.

The enzymes may be selected from cellulase, glucoamylase, amylase, pectinase, xylanase, or mixtures thereof; preferably a mixture of cellulase, glucoamylase, amylase, pectinase and xylanase. When a mixture of enzymes is used, the mixture is adjustable to account for the composition of the waste material in an in line series of tanks. For instance, the quantity of amylase may be adjusted to account for the starch, e.g. potato, content of the feedstock.

The slurry formed in step a. and/or step b. may be stored and/or transported to another location before the next step.

The liquid formed in step c. and/or the ethanol-water mixture formed in step d. may be stored and/or transported to another location before the next step.

The ethanol generated in step e. may be further treated to form a fuel selected from: diesel, fuel for an ethanol fuel cell, aviation fuel (sustainable aviation fuel), marine fuel or a substitute fossil fuel.

The method may not use acid hydrolysis. For instance, the method may not use acid hydrolysis to produce sugars from the slurry.

The waste material may be faeces, such as one or more of horse, cattle, pig, sheep, chicken, and turkey faeces, particularly chicken faeces.

The invention also relates to an apparatus for treating waste material according to any of the above methods.

The apparatus for treating waste material may comprise separate vessels for each of steps a-e.

The apparatus for treating waste material may comprise means for heating the slurry produced in step a. and/or means for reducing the temperature of the slurry produced in step a.

The apparatus for treating waste may comprise means for transferring heat energy to the slurry produced in step a. from the slurry prior to step b., preferably wherein the means for transferring heat energy is a heat exchanger.

The apparatus for treating waste material may be on one or more vehicles, for instance on or in a ship/tanker or across several vehicles.

The apparatus may be modular in design so that each stage may be performed separately or end-to-end as a continuous process.

The apparatus for treating waste material may be in one or more containers, such as in one or more shipping containers. For instance, the apparatus may be in a plurality of shipping containers, optionally wherein each container comprises the apparatus for performing a single step of the method. The containers may be connected end-to-end to form a single apparatus.

The use of household waste, such as food waste, garden waste, waste vegetation and cardboard, etc. to generate bioethanol diverts waste from landfill, compost heaps and incinerators, and so reduces landfill and CO₂ emissions from composting or incineration. Also, using MSW avoids using crops as a source of bioethanol.

High-quality ethanol (98.7 wt. %), is achievable with the apparatus and method of the invention, depending on the waste mix in the feedstock. Also, large volumes of bioethanol may be generated from MSW using the invention. Indeed, a pilot plant produced about 300 litres of bioethanol per tonne of pre-treated MSW.

The conversion of MSW to ethanol relies on enzymes whereas previous processes use acid hydrolysis, which relies on harmful corrosive substances with obvious environmental disadvantages.

The invention may be performed as a continuous, non-stop in line process, avoiding the need for storage or transport of any of the generated materials. This also allows for the recirculation of heat energy between stages of the process for increased thermal efficiency. Additionally, materials generated through the process may be used at earlier stages in the process to reduce costs. For instance, lignin generated during the process may be used as fuel for the apparatus or method, thus reducing waste and running costs. Also, CO₂ generated during the process may be used earlier in the process to aid in the breakdown of MSW by producing carbonic acid. Thus, the process may be optimised to minimise waste products and energy input.

The invention may capture any generated CO₂ to avoid emissions and also sell the captured CO₂ for additional revenue.

The materials generated throughout the process may, be stored or transported between steps. Thus, the invention may be performed across one or more vehicles, e.g., on or in a ship/tanker or across several vehicles, such as several trucks. This allows the apparatus or process to be brought to the MSW for preparation of bioethanol at source. The invention may also be performed across one or more locations, for instance allowing the conversion of MSW at source into one of the interim products, such as the ethanol-water mixture, transporting the interim product to another location for additional processing.

The invention scales readily, with small-scale apparatus being highly profitable and capacity easily expandable, e.g., through the addition of parallel lines or larger vessels.

The invention may be performed without using pressurised vessels for improved safety.

The fermentative production of bioethanol is known. WO2008025522A1 relates to a method of producing bioalcohol, in particular ethanol or butanol, from biomass, in which the biomass is comminuted, the remaining biomass is fed to a fermentation and the alcohol is obtained from the product of the fermentation, insoluble components and/or non-fermentable sugars being separated off from the biomass before the fermentation and/or yeast and bacteria are separated off after the fermentation.

The production of electricity can also be achieved with the use of gas turbine driven generators, using some of the ethanol, to provide the electrical requirement for the plant. This has the additional advantage that the energy in the exhaust gasses from the gas turbine can also contribute to the heating requirement of the plant.

Alternative methods of electrical production can be achieved by the use of direct ethanol fuel cells, providing a three-fold contribution to the production plant of electricity, water and heat. The electric power industry has generally been looking toward the use of fuel cells in relatively large electrical power generating applications. Power generation by fuel cells offers the advantages of high efficiency and low environmental emissions. Thus, fuel cells may offer a more economical means of power production than other existing power producing technologies.

It is shown from the foregoing description, that the bioethanol produced by the present invention can be used in a wide variety of ways. A particular plant may focus on a particular process, e.g., generating electricity from a fuel cell, or the production of SAF. The skilled person may implement this using one of the techniques described or referenced, or other techniques known in the art or as may be developed.

All refences to wt. % refer to weight percent by mass.

The inventions described and claimed herein are not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. Various references are cited herein, the disclosures of which are incorporated by reference in their entireties. The present invention is further described by the following examples which should not be construed as limiting the scope of the invention.

### EXAMPLE

MSW was obtained from household waste and sorted into feedstock comprising 85 wt. % carbohydrates, such as office waste (e.g., paper and card), garden waste and kitchen waste; 3 wt. % non-fermentable dissolve solids (NFDs); 4 wt. % protein solution; 6 wt. % C5 sugar solution; 1 wt. % other polymers and 1 wt. % oil/fat.

### Pre-Treatment Stage

The feedstock was reduced to a slurry with a particle size of about 1-3 mm using an automatic ‘self-adjusting’ hydraulic rota blade and the addition of water, if necessary. The reduction of the feedstock to a slurry of small particles increased the surface area of the feedstock, which helped the enzymes breakdown the lignocellulose in the feedstock.

The slurry was then heated to above the boiling point of water using heat exchangers and steam generated from a boiler. The boiler may use the lignin waste product generated in the process as fuel, thus ensuring an efficient process that minimises waste and carbon dioxide production. Increasing the temperature exposed the carbohydrate hidden in the lignocellulosic core, composed of cellulose and hemicellulose encased in a lignin matrix (recalcitrant), assisting the enzymes in the saccharification stage.

Heating the slurry to 138° C. was found to be the optimum temperature.

The temperature of the slurry was then reduced to a temperature to suit the enzymes using heat exchangers, and the pH level of the system was adjusted to suit the enzyme activity (pH dosing).

### Saccharification Stage

The slurry was then subject to enzymatic saccharification where the carbohydrate polymers were converted into sugar monomers, such as glucose, xylose and fructose.

The slurry was loaded into a hydrolysis vessel, diluted with water, if necessary, and the enzyme mixture added. The enzyme mixture is a blend of pectinase, xylanase, cellulose, amylase and glucoamylase. The relative amounts and total amounts of these enzymes is adjustable to accommodate different feedstocks, e.g., greater balance of food waste or garden waste, and seasonal variation.

Saccharification was optimized for enzyme activity and so occurred at about pH 5 and at 40-42° C. in an insulated tank ensuring that the temperature did not drop below 35° C. The saccharification time may be adjusted to accommodate seasonal variation and takes about 5-10 hours. The sugar levels were monitored throughout the process.

### Separation Stage

The liquid and solid waste from the slurry were separated using a centrifuge rotating at high speed (4000 g, 15 mins). The solid waste (lignin) collected during the separation stage was used to fuel the boiler in the pre-treatment stage. The liquid obtained from the separation stage was a sugar-water mixture.

### Fermentation Stage

The sugar-water mixture was converted to an ethanol-water mixture by fermentation using a turbo-yeast over 24 hours in highly insulated tanks that maintained the temperature at 46° C., for optimal fermentation. CO₂ was produced during fermentation and was captured and processed using a CO₂ liquefaction plant. The captured CO₂ may be sold or used to produce carbonic acid, to be added earlier in the process to lower the pH. In a typical process, the ethanol-water mixture had an ethanol concentration of 11 wt. %.

A Cleaning-in-Place (CIP) system automatically cleaned the entire system, i.e., the interior surfaces of pipes, vessels, and equipment, to ensure that the lines and vessels did not clog up and the system efficiency maintained.

### Purification Stage

Ethanol and water form a minimum boiling azeotrope, preventing purification beyond about 95% ethanol via simple distillation. High purity ethanol was recovered from the 11 wt. % ethanol-water mixture using a distillation column and a bespoke zeolites system. First, a heat exchanger increased the temperature of the mixture to 102° C., then pressure swing distillation using acetone was used to circumvent the azeotropic point. The output of the distillation column was a 95.7 wt. % ethanol mixture, which then proceeded through a zeolite system, where it was dehydrate via adsorption to provide fuel-grade ethanol (98.7 %).

The zeolites were regenerated by heating. The water distilled by the distillation column was used to increase the temperature of the incoming ethanol-water mixture from the fermentation tanks, thereby reducing energy consumption. The water was then passed through a reverse osmosis system to give distilled water, which may be used in the process, for instance it may be added during the pre-treatment stage.

### Sustainable Aviation Fuel (SAF)

The ethanol generated by the above method may be converted into SAF by:
i. dehydration of the ethanol to ethylene,
ii. oligomerisation of the ethylene into higher olefins, e.g., C₄-C₂₀ olefins;
iii. hydrogenation of the olefins to give paraffin,
iv. distillation of the paraffin to give gasoline (C₆-C₈), aviation (C₉-C₁₆) and diesel (C₁₆-C₂₂) range products.

This additional processing may be carried out directly on the ethanol generated by the above method, e.g., in an in-line process, or subsequently following storage or transport.

1. A method for treating waste material comprising the steps of:
a. converting the waste material into a slurry, e.g., by adding water;
b. adding enzymes to the slurry and allowing the enzymes to extract sugars from the slurry by enzyme saccharification;
c. separating the liquid content of the slurry from the solid waste content to give a liquid;
d. converting the liquid to an ethanol-water mixture;
e. concentrating or separating the ethanol-water mixture to give ethanol.

2. The method of claim 1, wherein the waste material is processed into a slurry in step a., wherein the particle size is reduced to less than about 10 mm, optionally less than about 5 mm, preferably about 1-3 mm.

3. The method of claim 1, wherein prior to step b. the slurry is heated to above the boiling point of water; for instance, to 100 to 175° C., optionally 125 to 150° C., preferably about 138° C., to assist in breaking down the slurry.

4. The method of claim 1, wherein prior to step b. the slurry is heated to 60 to 125° C., optionally 70 to 100° C., preferably about 85° C., to assist in breaking down the slurry.

5. The method of claim 1, wherein the temperature of the slurry is increased or reduced to about 40-42° C., optionally using heat exchangers, prior to adding the enzymes in step b.

6. The method of claim 1, wherein the pH of the slurry is adjusted to suit the enzymes, for instance to pH 5, before, during or after adding the enzymes.

7. The method of claim 1, wherein in step c. the liquid content of the slurry is separated from the solid waste content using a centrifuge.

8. The method of claim 1, wherein in step d. the liquid is converted into an ethanol-water mixture using yeast, e.g., using a turbo-yeast.

9. The method of claim 1, wherein the CO₂ generated in step d. during the conversion of the liquid to an ethanol-water mixture is captured.

10. The method of claim 9, wherein the captured CO₂ is used during subsequent methods to prepare carbonic acid which is used to lower the pH during subsequent methods, or wherein the method uses CO₂ captured during an earlier method to prepare carbonic acid, which is used to lower the pH.

11. The method of claim 1, wherein in step e. the ethanol-water mixture is concentrated using distillation and/or a zeolite column.

12. The method of claim 1, wherein the enzyme saccharification in step b. is allowed to occur over 5-10 hours; and/or at 40-42° C., and/or at pH 5.

13. The method of claim 3, wherein the solid waste content obtained in step c. is used as fuel to heat the process during subsequent methods or wherein the solid waste content generated during an earlier method is used to heat the process.

14. The method of claim 1, wherein the enzymes are selected from cellulase, glucoamylase, amylase, pectinase, xylanase, or mixtures thereof; preferably a mixture of cellulase, glucoamylase, amylase, pectinase and xylanase.

15. The method of claim 14, wherein a mixture of enzymes is used and the mixture is adjustable to account for the composition of the waste material in an in line series of tanks.

16. The method of claim 1, wherein the slurry formed in step a. and/or step b. is stored and/or transported to another location before the next step.

17. The method of claim 1, wherein the liquid formed in step c. and/or the ethanol-water mixture formed in step d. is stored and/or transported to another location before the next step.

18. The method according to claim 1, wherein the ethanol generated in step e. is further treated to form a fuel selected from: diesel, fuel for an ethanol fuel cell, aviation fuel (sustainable aviation fuel), marine fuel or a substitute fossil fuel.

19. The method according to claim 1, wherein the method does not use acid hydrolysis.

20. An apparatus for treating waste material according to claim 1.

21. The apparatus for treating waste material according to claim 20, comprising separate vessels for each of steps a-e.

22. The apparatus for treating waste material according to claim 20, comprising means for heating the slurry produced in step a. and/or means for reducing the temperature of the slurry produced in step a.

23. The apparatus for treating waste material according to claim 20, comprising means for transferring heat energy to the slurry produced in step a. from the slurry prior to step b., preferably wherein the means for transferring heat energy is a heat exchanger.

24. The apparatus for treating waste material according to claim 20, wherein the apparatus is on one or more vehicles, for instance on or in a ship/tanker or across several vehicles.

25. The apparatus for treating waste material according to claim 20, wherein the apparatus is in one or more containers, (e.g. one or more shipping containers), preferably the apparatus is in a plurality of shipping containers, more preferably wherein each container comprises the apparatus for performing a single step of the method, yet more preferably the containers are connected end-to-end to form a single apparatus.
